SEQUENCE LISTING (2 sequences)

SEQ ID NO: 1 - RNA, 19 nt, Artificial Sequence
  Description of Artificial Sequence Synthetic oligonucleotide
cggucaaaauucgagcaga

SEQ ID NO: 2 - RNA, 19 nt, Artificial Sequence
  Description of Artificial Sequence Synthetic oligonucleotide
gcauuuccuggagaguaca

(19) United States
(12) Patent Application Publication
(10) Pub. No.: US 2014/0364453 A1
(43) Pub. Date: Dec. 11, 2014

(54) SALUBRINAL-DRIVEN ATTENUATION OF MALIGNANT PHENOTYPES OF 4T1 BREAST CANCER CELLS

(71) Applicant: Indiana University Research and Technology Corporation, Indianapolis, IN (US)

(72) Inventors: Hiroki Yokota, Carmel, IN (US); Kazunori Hamamura, Indianapolis, IN (US)

(73) Assignee: Indiana University Research and Technology Corporation, Indianapolis, IN (US)

(21) Appl. No.: 14/296,888

(22) Filed: Jun. 5, 2014

Related U.S. Application Data
(60) Provisional application No. 61/831,549, filed on Jun. 5, 2013.

(51) Int. Cl. A61K 31/4706; A61K 31/155

(52) U.S. Cl. CPC A61K 31/4706; A61K 31/155; A61K 2201/094
USPC 514/311; 514/632

(57) ABSTRACT

A method is disclosed for the in vivo treatment of a breast cancer tumor utilizing a therapeutically effective amount of an agent that inactivates the Rac1 GTPase and results in a reduction in cancer cell invasion, reduced cancer cell motility, and reduced volume and weight of the tumor. Agents that can be used in this treatment include salubrinal, guanabenz, and combinations thereof. The agents additionally have proven useful in reducing the development of osteoclastogenesis and reducing bone metastasis.

### CROSS-REFERENCES TO RELATED APPLICATIONS

This application claims the benefit of U.S. Provisional Patent Application No. 61/831,549, filed Jun. 5, 2013, and entitled SALUBRINAL-DRIVEN ATTENUATION OF MALIGNANT PHENOTYPES OF 4T1 BREAST CANCER CELLS, which is incorporated herein by reference.

### BACKGROUND

Breast cancer accounts for about 25% of all cancers in women, and approximately 20% of breast cancer patients are likely to develop metastatic tumors in distant organs such as the lungs, liver, brain, and bone. In particular, bone is the most common site for metastasis of breast cancer. Certain strategies for the treatment of bone metastasis includes administration of bisphosphonates (e.g., zoledronate, pamidronate, and alendronate) and anti-receptor activator of nuclear factor kappa-B ligand (RANKL) antibody (Denosumab), which block bone resorption and improve the skeletal morbidity. Bisphosphonates are the most commonly used medication to reduced bone resorption, bone destruction, and tumor growth. However, it does not stimulate bone formation and it often exhibits side effects such as joint inflammation and avascular osteonecrosis of the jaw. Denosumab is reported to reduce bone metastasis, but its effect on tumor growth have not been elucidated. However, this treatment does not induce regression of the established bone metastasis.

Other therapeutic strategies depend heavily on the expression levels of three marker genes such as estrogen receptor (ER), progesterone receptor (PgR), and human epidermal growth factor receptor-2 (HER2). When cancer cells exhibit a high expression level of ER and/or PgR, hormonal treatments would be a viable option. For cancer cells with an overexpressed level of HER2, treatments with HER2-targeted drugs such as trastuzumab and lapatinib are potentially effective. A lack of expression of all three gene products, however, defines a triple negative breast cancer (TNBC) that presents a challenge in prognosis and requires a novel treatment option.

This study will focus on the metastasis of breast tumors to bone and will examine a therapeutic potential of salubrinal and guanabenz using a murine model of metastatic breast cancer to minimize bone metastasis and attenuate the cancer's malignant phenotype and tumor growth. In addition, treatments for triple negative breast cancer will be studied.

### SUMMARY

Salubrinal is a synthetic chemical agent that is known to alter a cellular fate through elevation of the phosphorylation level of eukaryotic translation initiation factor 2α (eIF2α). Guanabenz similarly elevates the phosphorylation level of eIF2α.

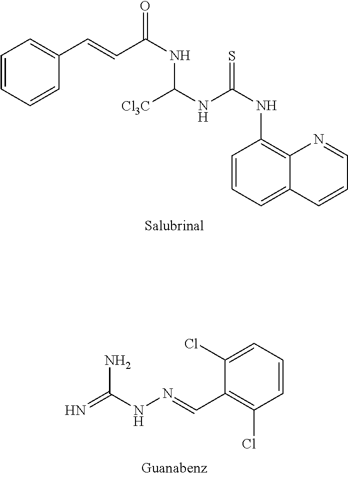

In this study, research focused on the question of whether administration of salubrinal and/or guanabenz attenuate malignant phenotypes of triple negative breast cancer cells (TNBCs) that lack an estrogen receptor, a progesterone receptor, and the human epidermal growth factor receptor-2 as well as tumor growth by elevating the level of phosphorylated eIF2α.

The use of salubrinal and guanabenz as an inhibitory agent of dephosphorylation of eIF2α, to provide an elevated level of phosphorylated eIF2α and attenuate malignant phenotypes of triple negative breast cancer cells (TNBCs) that lack estrogen receptor, progesterone receptor, and human epidermal growth factor receptor-2 was studied. Effects of salubrinal and guanabenz on in vitro phenotype of 4T1 mammary tumor cells and MDA-MB-231 human breast cancer cells, were determined and their effects on in vivo tumor growth using BALB/c mice injected with 4T1 cells evaluated. The results revealed that these agents block the proliferation and survival of 4T1 and MDA-MB-231 cells, as well as their invasion and motility. Silencing eIF2α revealed that eIF2α is involved in the reduction in invasion and motility. Furthermore, salubrinal-driven inactivation of Rac1 was suppressed in the cells treated with eIF2α siRNA, and treatment with Rac1 siRNA reduced cell invasion and motility. In vivo assay revealed that subcutaneous administration of salubrinal reduced the volume and weight of tumors induced by 4T1 cells. Collectively, the results indicate that these agents can attenuate malignant phenotype and tumor growth of breast cancer cells through the eIF2α-mediated Rac1 pathway. This study supports the use of eIF2α-mediated Rac1 regulation in suppressing the growth and metastasis of breast cancer.

One aspect of this present disclosure includes an in vivo method for treating a malignant tumor by providing a mammal exhibiting a malignant tumor; and treating the mammal with a therapeutically effective amount of a pharmaceutically acceptable agent capable of inactivating the Rac1 GTPase. A malignant tumor can include a malignant tumor associated with breast cancer, particularly a triple negative form of breast cancer. Suitable agents include salubrinal, guanabenz, and/or combinations thereof. One result of this treatment includes a reduction in cancer cell invasion, reduced cancer cell motility, and reduced volume and weight of the tumor. Agents can be administered orally or by injection, osmotic pump, IV administration, ingestion, dermal application, and inhalation. Injection of the agent can involve injection at the site of the tumor.

A further aspect of the present disclosure includes an in vivo method for treating a malignant tumor by providing a human exhibiting symptoms of breast cancer and treating the human with a therapeutically effective amount of a pharmaceutically acceptable agent including a compound capable of elevating the phosphorylation level of eIF2α within the human. Therapeutically effective agents include salubrinal, guanabenz and a combination thereof. The method is effective against tripple negative forms of breast cancer. Agents selected can be administered orally or by injection, osmotic pump, IV administration, ingestion, dermal application, and inhalation.

A still further aspect of the present disclosure includes an in vivo method for reducing the development osteoclastogenesis in a human by treating the human with a therapeutically effective amount of a pharmaceutically acceptable agent including a compound selected from the group consisting of salubrinal, guanabenz and a combination thereof. The administration of these agents similarly reduces and/or prevents bone metastasis.

### BRIEF DESCRIPTION OF THE DRAWINGS

FIG. 1 illustrates the inhibitory effects of salubrinal and guanabenz in proliferation and survival of 4T1 cells. The single and double asterisks indicate p<0.05 and p<0.01, respectively. CN: control, Sal: salubrinal, Gu: guanabenz, and FCS: fetal calf serum. (A) Relative cell growth in response to 10, 20, or 50 μM salubrinal. (B) Relative cell growth in response to 5, 10, 20, or 50 μM guanabenz. (C) Dose-dependent elevation of cleaved caspase 3 to 10-50 μM salubrinal in the absence of serum. (D) Dose-dependent elevation of cleaved caspase 3 to 5-50 μM guanabenz in the absence of serum. (E) Reduced elevation of cleaved caspase 3 to salubrinal in the presence of 10% serum. (F) Reduced elevation of cleaved caspase 3 to guanabenz in the presence of 10% serum.

FIG. 2 illustrates the dose-dependent reduction in invasion and reduction in motility of 4T1 cells. The single and double asterisks indicate p<0.05 and p<0.01, respectively. CN: control, Sal: salubrinal, and Gu: guanabenz. (A) Percent of the invaded cells in response to 10, 20 or 50 μM salubrinal in the absence of serum. (B) Percent of the invaded cells in response to 5, 10, 20 or 50 μM guanabenz in the absence of serum. (C) Percent of the invaded cells in response to salubrinal in the presence of 10% serum. (D) Reduction in cell motility by 20 or 50 μM salubrinal in 24 h in the absence of serum. (E) Reduction in cell motility by 20 or 50 μM guanabenz in 24 h in the absence of serum. (F) Sustained reduction in cell motility by salubrinal in the presence of 10% serum.

FIG. 3 illustrates the inhibitory effects of salubrinal in proliferation, invasion, survival, and motility of MDA-MB-231 cells. The single and double asterisks indicate p<0.05 and p<0.01, respectively. CN: control, and Sal: salubrinal. (A) Relative cell growth in response to 20 or 50 μM salubrinal. (B) Reduction in cell invasion by 20 or 50 μM salubrinal in 24 h in the absence of serum. (C) Dose-dependent elevation of cleaved caspase 3 to 20 or 50 μM salubrinal in the absence of serum. (D & E) Reduction in cell motility by 20 or 50 μM salubrinal in 24 h in the presence of 10% serum.

FIG. 4 illustrates the involvement of eIF2α in salubrinal-driven reduction in cell invasion and motility of 4T1 cells. Salubrinal was given at 20 or 50 μM. The single and double asterisks indicate p<0.05 and p<0.01, respectively. Note that NC designates the samples treated with the non-specific control siRNA. Sal: salubrinal. (A) Reduction in the protein level of eIF2α by RNA interference. (B) Partial suppression of salubrinal-driven reduction in cell invasion by eIF2α siRNA. (C and D) Suppression of salubrinal-driven reduction in cell motility (elevation of the wound areas) by eIF2α siRNA.

FIG. 5 illustrates the FRET-based detection of inactivation of Rac1 GTPase in response to 20 μM salubrinal. In live cell imaging, the color bar represents the emission ratio of YFP/CFP, an index of Rac1 activation and the arrow indicates the commencement time (t=0) for administration of salubrinal. Scale bar=10 μm. (A) Normalized Rac1 activity in 4T1 cells. (B) Normalized Rac1 activity in MDA-MB-231 cells. (C) Normalized Rac1 activity in the presence of eIF2α siRNA or non-specific control (NC) siRNA in 4T1 cells.

FIG. 6 illustrates the reduction in cell invasion and motility of 4T1 cells by RNA interference with Rac1 siRNA. Note that NC designates the samples treated with the non-specific control siRNA. The double asterisk indicates p<0.01. (A) Reduction in the protein level of Rac1 by RNA interference. (B) Reduction of cell growth by Rac1 siRNA. (C) Increase in cleaved caspase 3 by Rac1 siRNA. (D) Reduction in cell invasion by Rac1 siRNA. (E and F) Reduction in cell motility by Rac1 siRNA.

FIG. 7 illustrates the inhibitory effects of salubrinal on tumor growth with 4T1 cells. (A) Images of harvested tumors in the control and salubrinal-treated groups. (B-C) Comparison of the tumor volume and weight, respectively. The circles and triangles represent the values for the control and salubrinal-treated mice, respectively. The horizontal bars indicate the mean values for each group. N=17 for control and N=18 for the salubrinal-treated mice. Sal: salubrinal. (D) Proposed mechanism of salubrinal's action on breast cancer cells.

FIG. 8 illustrates the suppression of osteoclastogenesis by salubrinal and guanabenz. Primary mouse bone marrow cells were incubated with 10 or 20 μM salubrinal/guanabenz. (A) Reduced development of osteoclasts by administration of salubrinal and guanabenz for 3 days. (B) Reduction in TRAP-positive multi-nucleated cells by incubation with salubrinal and guanabenz for 3 days. (C) Suppression of NFATc1 protein expression by incubation with salubrinal and guanabenz for 2 days.

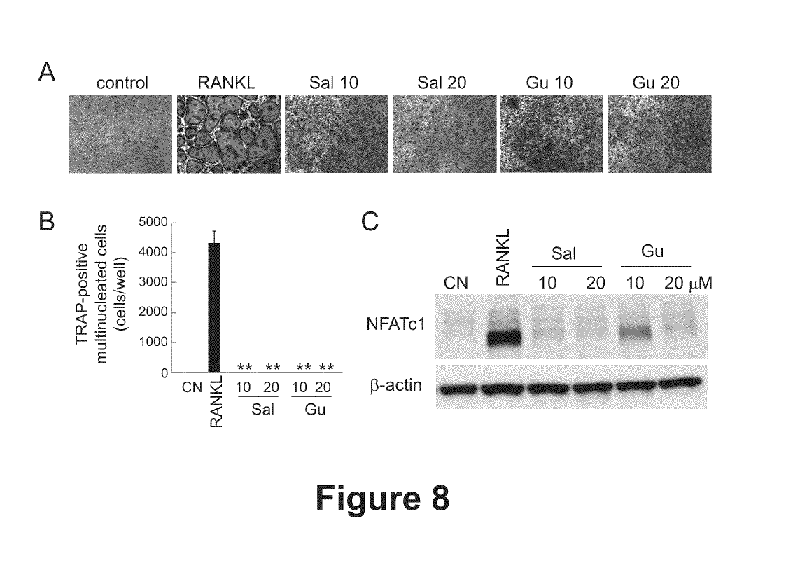

FIG. 9 illustrates the relative mRNA abundance in response to salubrinal and guanabenz. Administration of 10 μM salubrinal or guanabenz for 24 h reduced the mRNA levels of NFATc1, TRAP, OSCAR, c-fos, MMP9, and cathepsin K in primary mouse bone marrow cells.

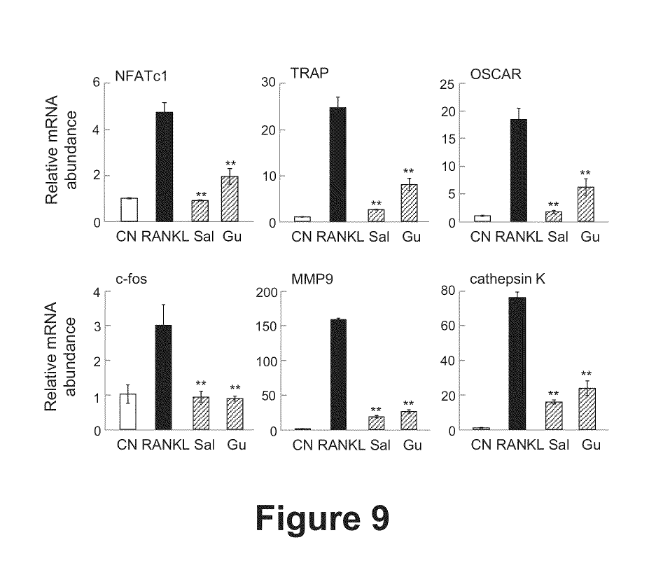

### DESCRIPTION

Before the present methods, implementations and systems are disclosed and described, it is to be understood that this invention is not limited to specific synthetic methods, specific components, implementation, or to particular compositions, and as such may, of course, vary. It is also to be understood that the terminology used herein is for the purpose of describing particular implementations only and is not intended to be limiting. Neither are mechanisms which have been provided to assist in understanding the disclosure meant to be limiting.

As used in the specification and the claims, the singular forms “a,” “an” and “the” include plural referents unless the context clearly dictates otherwise. Ranges may be expressed in ways including from “about” one particular value, and/or to “about” another particular value. When such a range is expressed, another implementation may include from the one particular value and/or to the other particular value. Similarly, when values are expressed as approximations, for example by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Similarly, “typical” or “typically” means that the subsequently described event or circumstance often though may not occur, and that the description includes instances where said event or circumstance occurs and instances where it does not.

Various environmental stresses such as oxidation, nutrient deprivation, radiation, and stress to the endoplasmic reticulum induce the integrated stress response in which the elevated phosphorylation level of eukaryotic translation initiation factor 2α (eIF2α) may stimulate cellular apoptosis. In response to mild stresses, the phosphorylation of eIF2α attenuates translational efficiency and activates the pro-survival signaling. However, in response to severe stress, the phosphorylated eIF2α (eIF2α-p) promotes apoptosis. Salubrinal is known to elevate the level of eIF2α-p and is considered as a cytoprotective agent as well as an agent to stimulate apoptosis depending on the stress environments and cell types. For instance, salubrinal has been reported to protect against tunicamycin induced cardiomyocyte apoptosis. On the contrary, administration of salubrinal to leukemic and chondrosarcoma cells was reported to stimulate apoptosis.

The current study addresses the question: does administration of salubrinal attenuate the malignant in vitro phenotype of TNBCs? If yes, what is a mechanism of salubrinal's action and does its administration to mice injected with TNBCs suppress in vivo tumor growth? In response to administration of salubrinal, the in vitro phenotype of 4T1 mammary tumor cells and MDA-MB-231 human breast cancer cells were studied. In addition, the effect of guanabenz, another synthetic drug known to elevate eIF2α-p by inhibiting de-phosphorylation of eIF2α-p was also studied. In order to examine the involvement of eIF2α, silencing by RNA interference was conducted using siRNA specific to eIF2α. In addition, the potential linkage between regulation of eIF2α and Rac1 GTPase in cell invasion and motility was evaluated using siRNA specific to Rac1 GTPase. A FRET (fluorescence resonance energy transfer) technique was employed and Rac1 activity in response to salubrinal evaluated. To test the effects of salubrinal in vivo, 4T1 mammary tumor cancer cells were injected to mice and suppression of tumor growth was analyzed.

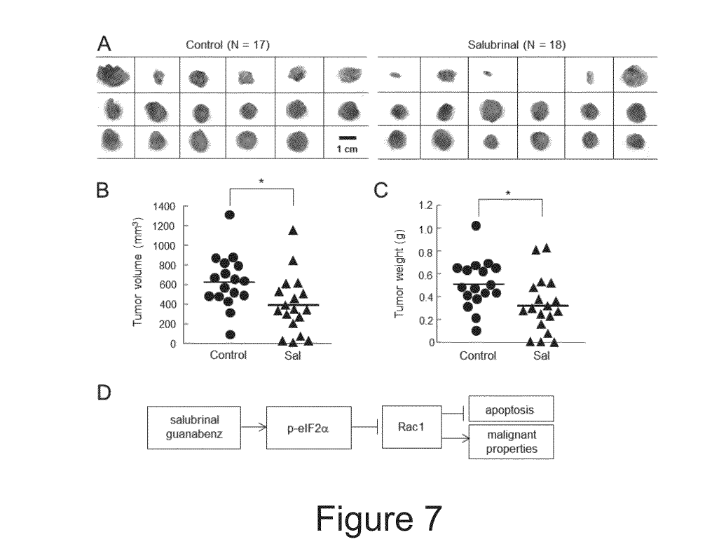

The results of these studies demonstrate that salubrinal has inhibitory effects on the malignant phenotypes of 4T1 mammary tumor cells and MDA-MB-231 breast cancer cells that hold a triple negative phenotype. Salubrinal significantly reduced cellular proliferation, invasion, and migration, although it did not alter cellular adhesion to surfaces coated with poly-L-lysine, type I collagen, fibronectin, or laminin. The inhibitory effects were commonly observed in response to salubrinal and guanabenz, both of which can elevate the level of p-eIF2α. RNA silencing with eIF2α siRNA eliminated salubrinal driven reduction in Rac1 and RNA silencing with Rac1 siRNA attenuated malignant phenotypes as seen in the responses to salubrinal. Furthermore, in vivo tumor size and weight in 4T1 cells injected mice were significantly reduced by daily administration of salubrinal (FIG. 7D).

The elevated level of cleaved caspase 3 observed indicated that cellular apoptosis was stimulated by salubrinal. An increase in apoptotic death was more significant in the absence of FBS in the medium than that with FBS, suggesting that potency of salubrinal is enhanced in a nutrient poor environment. Previous studies reported that salubrinal could induce either the stimulatory or inhibitory effects on cellular death through modulation of the level of p-eIF2α. Our results are consistent with a concept that in an abnormal growth condition such as in a solid tumor without well-developed vasculature, the elevation of p-eIF2α leads to a pro-apoptotic pathway.

Rac1 GTPase is a regulator of various cellular processes, including cell cycle, motility, invasion, and cell-cell adhesion. It is known to play a substantial role in the development of various cancers including breast cancer and pancreatic cancer. Rac-guanine nucleotide exchange factor (GEF) P-Rex1 is reported to be an essential stimulator of Rac1 activation, and P-Rex1 is reported to be activated by the phosphatidylinositide 3-kinases (PI3K) pathway. Furthermore, Rac1 can be activated through integrins, tyrosin-kinase receptors, and various stress factors including mechanical stimulation, and the stress to the endoplasmic reticulum. Because salubrinal was able to relieve the stress to the endoplasmic reticulum, it is plausible that eIF2α-mediated Rac1 suppression is linked to modulation of stress responses in 4T1 mammary tumor cells.

Because 4T1 and MDA-MB-231 cells are considered to be triple negative, salubrinal's action is clearly different from that of selective estrogen receptor modulators (SERMs) which target the estrogen receptor. Tamoxifen, for instance, is thought to act as an agonist at the bone and uterus and an antagonist at the breast. Unlike salubrinal, however, tamoxifen is not effective for estrogen receptor negative breast cancer cells. Regarding the involvement of Rac1 in response to salubrinal, it has been reported that inhibition of Rac1 using a pharmacological agent (EHT1864) decreases estrogen receptor levels and proliferation of both tamoxifen-sensitive and resistant cells. Although the reported study suggests that inhibition of Rac1 could be a therapeutic strategy for estrogen receptor positive cells, the current study indicates that salubrinal-driven reduction of Rac1 is also effective in attenuating malignant phenotypes of estrogen receptor negative cells. In this context, MDA-MB-231 cells used in this study have K-Ras mutation and are dependent on Rac1 for growth factor driven invasion and migration. Therefore, it is plausible that Rac1 activity can serve as a biomarker of response to salubrinal.

Approximately 20% of breast cancer patients are likely to develop metastatic tumors in distant organs such as the lungs, liver, brain, and bone. Bone is the most common site for metastasis of breast cancer. The current chemotherapy for the treatment of bone metastasis includes administration of SERMs such as tamoxifen, bisphosphonates (e.g., zoledronate, pamidronate, and alendronate), and anti-receptor activator of nuclear factor kappa-B ligand (RANKL) antibody (Denosumab). Bisphosphonates are the most commonly used medication to reduce bone resorption, bone destruction and tumor growth. However, it does not stimulate bone formation and it often exhibits side effects such as joint inflammation and avascular osteonecrosis of the jaw. Denosumab is reported to reduce bone metastasis, but it effects on tumor growth have to be elucidated. Salubrinal and guanabenz, which inhibit the de-phosphorylation of eIF2α, could potentially both stimulate osteoblastgenesis through upregulation of ATF4 and attenuate osteoclastogenesis through downregulation of nuclear factor of activated T-cells, cytoplasmic 1 (NFATc1). Because salubrinal was shown to stimulate the growth of new bone and enhance the healing of bone wound, its effects on tumor growth would have a significant impact on treatment of breast cancer and bone metastasis.

The current study demonstrates that an inhibitory agent of dephosphorylation of eIF2α potentially offers a novel therapeutic strategy for attenuating malignant phenotypes of triple negative breast cancer cells. It was shown to downregulate the activity of Rac1 through eIF2α mediated signaling. Results also demonstrate that it can prevent not only tumor growth but also bone resorption associated with metastasis to bone.

### EXPERIMENTAL

### Cell Culture

4T1 mouse mammary tumor cells and MDA-MB-231 human breast cancer cells were cultured in DMEM containing 10% fetal bovine serum and antibiotics (50 units/ml penicillin, and 50 μg/ml streptomycin; Life Technologies, Grand Island, N.Y., USA). Cells were maintained at 37° C. and 5% CO₂ in a humidified incubator. Responses to administration of 10-50 μM salubrinal or 5-50 μM guanabenz acetate (Tocris Bioscience, Ellisville, Mo., USA) were evaluated using assays for MTT, adhesion, invasion, and motility.

### MTT Assay:

Cells (5×10²/well) were seeded in 96-well plates, and the reduction of MTT to formazan was evaluated by measuring the absorbance at 570 nm with a plate reader (EL800, BioTek, Winooski, Vt., USA).

### Cell Adhesion Assay:

Ninety-six well plates were coated with poly-L-lysine, fibronectin, laminin (Sigma-Aldrich, St. Louis, Mo., USA), or type I collagen (BD Biosciences, Bedford, Mass., USA) for 2 h. The plates were then incubated with non-fat dry milk, followed by washing with PBS and serum-free culture medium. Cells (1×10⁴/well) were added on the plate, and after 30 min and 3 h the attached cells were stained with 0.04% crystal violet (Sigma-Aldrich) for 10 min at room temperature. The wells were washed with PBS, and DMSO was added. Absorbance at 550 nm was measured using the plate reader.

### In Vitro Invasion Assay:

An invasion assay was performed with a Boyden chamber with minor modifications of the usual procedure. In brief, Matrigel (BD Biosciences) was diluted with ice-cold PBS (100 μg/ml). Six-hundred μl of Matrigel was added to each filter (polyethylene terephthalate membrane, 8-μm pore size, 23.1 mm in diameter, Falcon) and left to polymerize overnight. Prior to assembling the chamber unit, the lower chamber (6-well plate, Falcon) was filled with culture medium consisting of salubrinal or guanabenz. Cells (1˜4×10⁵/well) were added to the culture medium with salubrinal or guanabenz in the upper chamber and incubated for 24 h. The cells on the filter surface were stained with Giemsa (Sigma-Aldrich) and the number of cells was counted under the microscope.

### Two-Dimensional Motility Assay:

To evaluate 2-dimensional motility, a wound healing scratch motility assay was carried out. In brief, cells were plated in 12-well plates or 6-cm dishes (Falcon) and on the next day, scratching was performed using a plastic tip. The areas newly occupied with cells in the scratched zone were determined every 3 h up to 24 h using images obtained by a microscope, which were scanned with Adobe Photoshop (CS2, Adobe Systems, San Jose, Calif., USA) and quantified with Image J.

### Western Blot Analysis:

Cells were lysed in a radioimmunoprecipitation assay (RIPA) buffer containing protease inhibitors (Santa Cruz Biotechnology, Santa Cruz, Calif., USA) and phosphatase inhibitors (Calbiochem, Billerica, Mass., USA). Isolated proteins were fractionated using 10-15% SDS gels and electro-transferred to Immobilon-P membranes (Millipore, Billerica, Mass., USA). The membrane was incubated for 1 h with primary antibodies followed by 45 min incubation with goat anti-rabbit or anti-mouse IgG conjugated with horseradish peroxidase (Cell Signaling, Danvers, Mass., USA). We used antibodies against eIF2α, caspase 3, cleaved caspase (Cell Signaling), Rac1 (Millipore), and β-actin (Sigma). Protein levels were assayed using a SuperSignal west femto maximum sensitivity substrate (Thermo Scientific, Waltham, Mass., USA), and signal intensities were quantified with a luminescent image analyzer (LAS-3000, Fuji Film, Tokyo, Japan).

Knockdown of eIF2α and Rac1 by siRNA:

Cells were treated with siRNA specific to eIF2α and Rac1 (Life Technologies). Selected target sequences for knockdown of eIF2α and Rac1 were: eIF2α, 5′-CGG UCA AAA UUC GAG CAG A-3′, and Rac1, 5′-GCA UUU CCU GGA GAG UAC A-3′; and As a nonspecific control, a negative siRNA (Silencer Select #1, Life Technologies) was used. Cells were transiently transfected with siRNA for eIF2α, Rac1, or control in Opti-MEM I medium with Lipofectamine RNAiMAX (Life Technologies). Six hours later, the medium was replaced by regular culture medium. The efficiency of silencing was assessed with immunoblotting 48 h after transfection.

### Fluorescence Resonance Energy Transfer (FRET):

To visualize Rac1 activity in response to salubrinal, FRET imaging was conducted using a cyan fluorescent protein (CFP)-yellow fluorescent protein (YFP) Rac1 biosensor. The filter sets (Semrock) were chosen for CFP excitation at 438±24 nm (center wavelength±bandwidth), CFP emission at 483±32 nm, and YFP emission at 542±27 nm. Time-lapse images were acquired at an interval of 5 min using a fluorescence microscope (Nikon, Tokyo, Japan). The level of Rac1 activity was determined by computing an emission ratio of YFP/CFP for individual cells using NIS-Elements software (Nikon).

### In Vivo Tumor Growth:

Experimental procedures were approved by the Indiana University Animal Care and Use Committee and were in compliance with the Guiding Principles in the Care and Use of Animals endorsed by the American Physiological Society. Five mice were housed per cage, and fed with mouse chow and water ad libitum. Thirty-five BALB/c female mice (6 weeks, Harlan Laboratories) were used. Mice received subcutaneous injection of 4T1 mouse mammary tumor cells (10⁶ cells in 100 μl PBS) to the abdomen on day 1. Twenty-five μg of salubrinal was administered subcutaneously into the area of cell injection every day, while the control animals received a vehicle. The animals were sacrificed on day 20, and the volume and weight of tumors were determined. The tumor volume was calculated as (long diameter)×(short diameter)²/2.

### Statistical Analysis:

Three or four-independent experiments were conducted and data were expressed as mean±S.D. For comparison among multiple samples, ANOVA followed by post hoc tests was conducted. Statistical significance was evaluated at p<0.05. The single and double asterisks and daggers indicate p<0.05 and p<0.01.

### Results:

### Inhibitory Effects of Salubrinal and Guanabenz in Proliferation and Survival of 4T1 Cells:

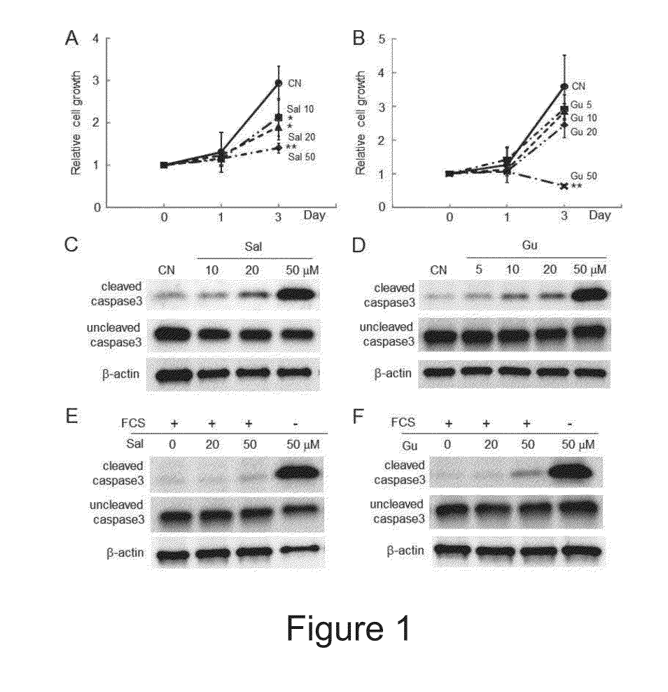

The MTT assay revealed that in response to 10, 20, and 50 μM salubrinal, the number of live 4T1 cells was reduced in a dose dependent manner (FIG. 1A). The number of live cells was also decreased by 50 μM guanabenz (FIG. 1B). Consistent with the MTT results, both salubrinal and guanabenz elevated the level of cleaved caspase 3 in the absence and presence of 10% serum in the culture medium, respectively (FIG. 1C-1F).

### Undetectable Changes in Cell Adhesion by Salubrinal and Guanabenz in 4T1 Cells

On the surface coated with poly-L-lysine, type I collagen, fibronectin, and laminin, the effects of salubrinal and guanabenz on cell adhesion were examined. The absorbance reading, which indicated the number of adherent cells on the coated surface, did not significantly change in the presence and absence of salubrinal and guanabenz 30 min and 3 h after cell incubation, respectively (data not shown).

### Dose-Dependent Reduction in Cell Invasion by Salubrinal and Guanabenz in 4T1 Cells:

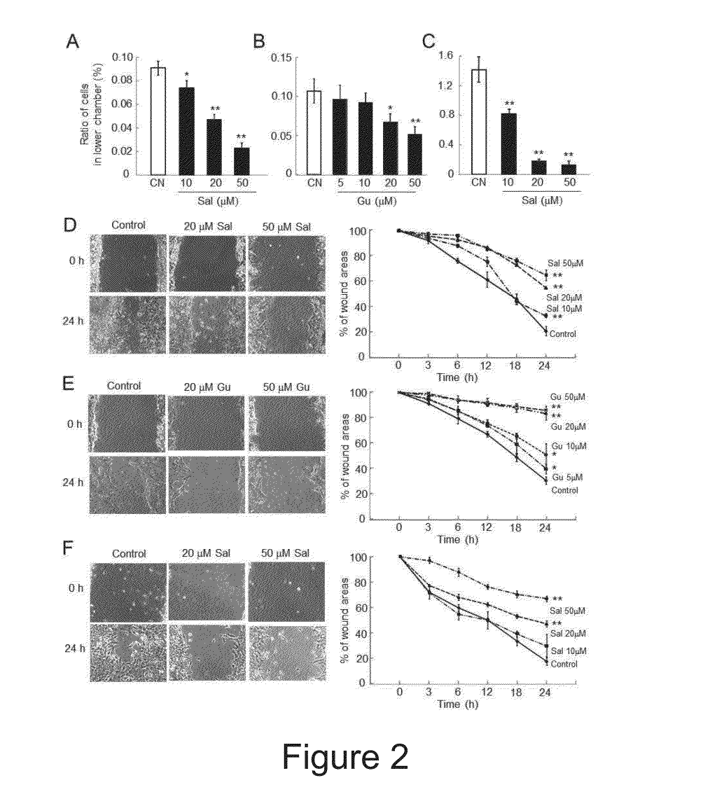

The number of cells invaded through the filter coated with Matrigel was significantly reduced by administration of salubrinal and guanabenz regardless of the presence of serum in the medium in a dose dependent manner (FIG. 2A-C).

### Reduction in Cell Motility by Salubrinal and Guanabenz in 4T1 Cells:

Using the scratch-wound assay, the wound area was determined as an indicator of cell motility in which a reduction in motility corresponded with a decrease in wound healing. In response to both salubrinal and guanabenz, cell motility was reduced in a dose dependent manner (FIG. 2D-F). The results with salubrinal were not affected by the presence of serum in the culture medium.

### Reduction in Proliferation, Invasion, Survival, and Motility in MDA-MB-231 Cells:

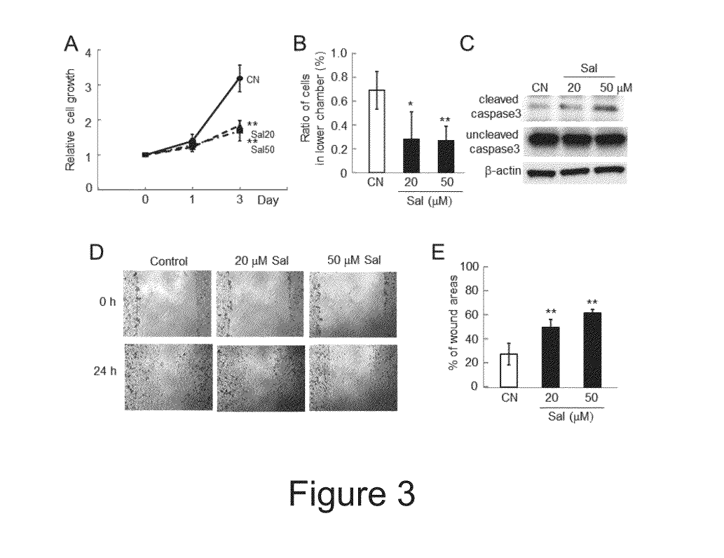

Consistent with the results in 4T1 mouse mammary tumor cells, a reduction was observed in proliferation, invasion, survival, and motility in MDA-MB-231 human breast cancer cells (FIG. 3).

Involvement of eIF2α in Salubrinal-Driven Reduction in Cell Invasion and Motility in 4T1 Cells:

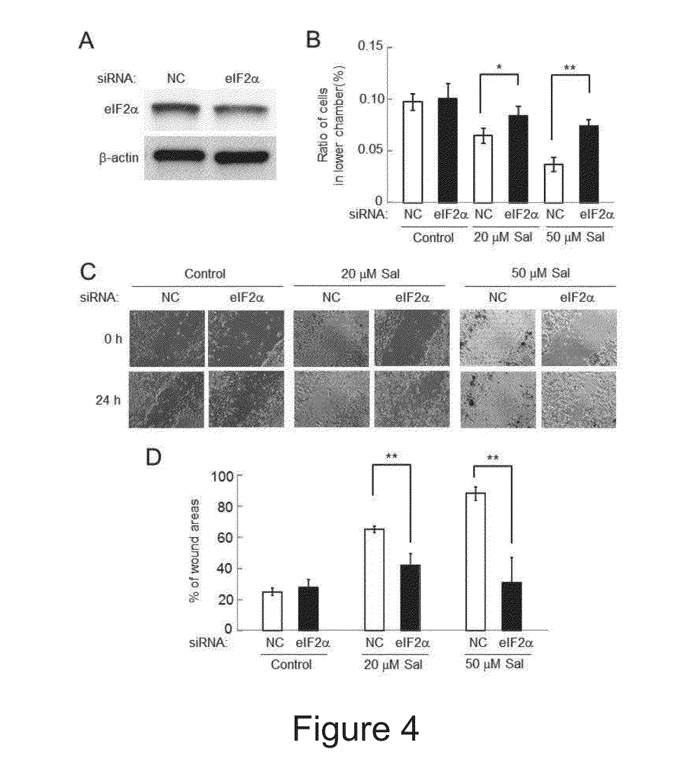

Cell invasion and motility in response to salubrinal were examined using the cells transiently transfected with eIF2α siRNA (FIG. 4A). Compared to the cells transfected with nonspecific control (NC) siRNA, salubrinal-driven reduction in cell invasion was significantly suppressed in the cells treated with eIF2α siRNA (FIG. 4B). However, in the control cells treated with NC siRNA, the cell invasion was reduced by salubrinal in a dose dependent manner. Furthermore, salubrinal-driven reduction in cell motility was also suppressed by eIF2α siRNA (FIGS. 4C & 4D).

### Inactivation of Rac1 GTPase by 20 MI Salubrinal in 4T1 Cells and MDA-MB-231 Cells:

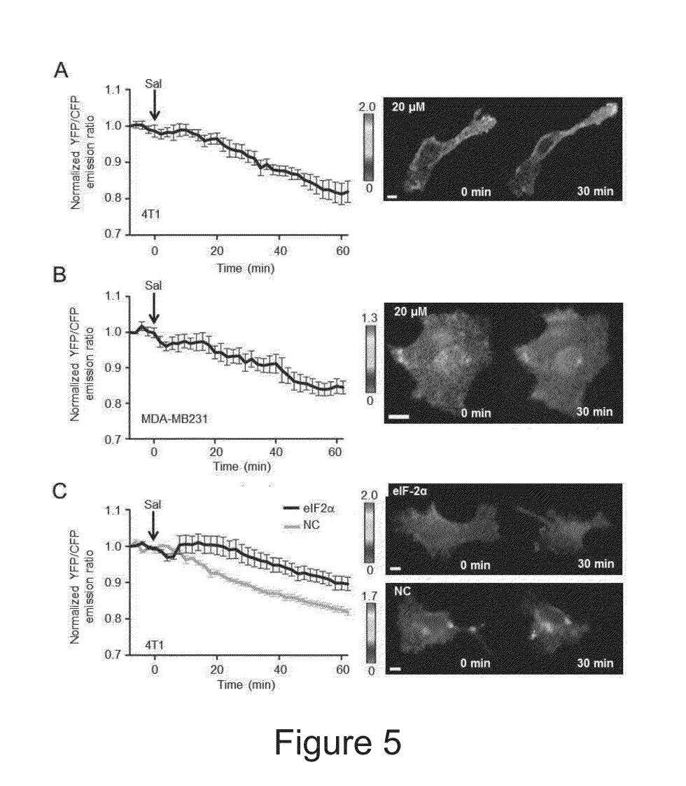

To evaluate thel involvement of Rac1 through eIF2α-mediated signaling, the effect of salubrinal on activity of Rac1 GTPase was examined using FRET-based single cell imaging. In response to 20 μM salubrinal, the emission ratio of YFP/CFP was decreased in 4T1 cells as well as MDA-MB-231 cells, indicating that administration of salubrinal reduced the activity level of Rac1 (FIGS. 5A & 5B). The FRET analysis was also conducted using 4T1 cells transfected with eIF2α siRNA, in which salubrinal-driven reduction in Rac1 activity was significantly suppressed (FIG. 5C).

Reduction in Cell Growth, Invasion and Motility by Rac1 siRNA in 4T1 Cells:

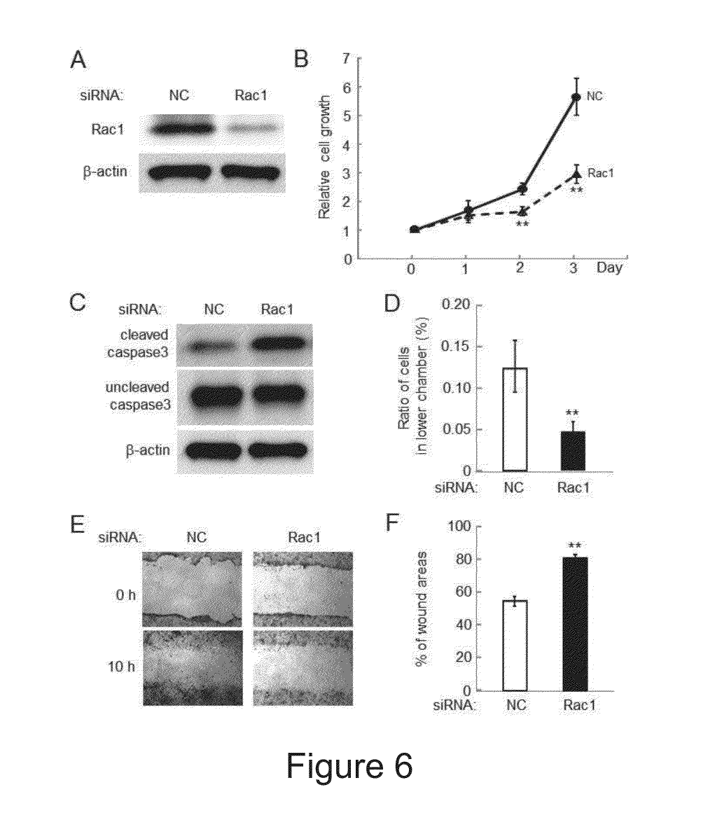

In order to examine the role of Rac1 in salubrinal-driven suppression of malignant phenotypes, cell growth, invasion and motility were examined using siRNA specific to Rac1 (FIG. 6A). The result revealed that reduction in the expression level of Rac1 lowered cell growth and elevated the level of cleaved caspase 3 (FIGS. 6B & 6C). Furthermore, silencing Rac1 decreased cell invasion as well as cell motility (FIG. 6D-6F).

### Inhibitory Effects of Salubrinal in the Volume and Weight of Tumors:

Using the in vivo mouse model, the effects of salubrinal on tumor growth were evaluated. A comparison of the tumors isolated from the control mice (N=17) and the salubrinal-treated mice (N=18) revealed that the tumor volume (control: 620.1±271.1 mm³; and salubrinal: 384.5±248.1 mm³) and weight (control: 0.51±0.21 g; and salubrinal: 0.32±0.24 g) were significantly larger in the control group than the salubrinal-treated group (FIGS. 7A & 7B).

While the disclosure has been illustrated and described in detail in the figures and foregoing description, the same is to be considered as illustrative and not restrictive in character, it being understood that only selected embodiments have been shown and described and that all changes, modifications and equivalents that come within the spirit of the disclosures described heretofore and/or defined by the following points of novelty are hereby fully disclosed.

1. An in vivo method for treating a malignant tumor comprising:
(a) providing a mammal exhibiting a malignant tumor and producing Rac1 GTPase; and
(b) treating the mammal with a therapeutically effective amount of a pharmaceutically acceptable agent capable of inactivating Rac1 GTPase.

2. The method of claim 1, wherein providing a mammal involves providing a human exhibiting a malignant tumor associated with breast cancer.

3. The method of claim 2, wherein providing a human involves providing a human exhibiting a malignant tumor associated with triple negative breast cancer.

4. The method of claim 3, wherein treating a human with a therapeutically effective amount of a pharmaceutically acceptable agent capable of inactivating the Rac1 GTPase involves treating the human with an agent selected from the group consisting of salubrinal, guanabenz and a combination thereof.

5. The method of claim 4, wherein treating a human with the therapeutically effective amount of a pharmaceutically acceptable agent capable of inactivating the Rac1 GTPase involves treating the human with salubrinal.

6. The method of claim 4, wherein treating a human with a therapeutically effective amount of a pharmaceutically acceptable agent capable of inactivating the Rac1 GTPase involves treating the human with guanabenz.

7. The method of claim 4, wherein treating a human with a therapeutically effective amount of a pharmaceutically acceptable agent capable of inactivating the Rac1 GTPase involves treating the human with a combination of salubrinal and guanabenz.

8. The method of claim 4, wherein treating a human with a therapeutically effective amount of a pharmaceutically acceptable agent capable of inactivating the Rac1 GTPase, provides at least one result selected from the group consisting of a reduction in cancer cell invasion, reduced cancer cell motility, reduced volume of the tumor and reduced weight of the tumor.

9. The method of claim 4, wherein treating a human with a therapeutically effective amount of a pharmaceutically acceptable agent capable of inactivating the Rac1 GTPase involves administering the agent by ingestion.

10. The method of claim 4, wherein treating a human with a therapeutically effective amount of a pharmaceutically acceptable agent capable of inactivating the Rac1 GTPase involves administering the agent at a site of the tumor.

11. The method of claim 4, wherein treating a human with a therapeutically effective amount of a pharmaceutically acceptable agent capable of inactivating the Rac1 GTPase involves administering the agent by a method selected from the group consisting of injection, osmotic pump, IV administration, dermal application, and inhalation.

12. An in vivo method for treating a malignant tumor associated with breast cancer comprising:
(a) providing a human exhibiting symptoms of breast cancer; and
(b) treating the human with a therapeutically effective amount of a pharmaceutically acceptable agent capable of elevating the phosphorylation level of eIF2α.

13. The method of claim 12, wherein treating a human with a therapeutically effective amount of a pharmaceutically acceptable agent involves treating the human with an agent selected from the group consisting of salubrinal, guanabenz and a combination thereof.

14. The method of claim 13, wherein providing a human exhibiting symptoms of breast cancer involves providing a human exhibiting a triple negative form of breast cancer.

15. The method of claim 14, wherein treating a human exhibiting symptoms of triple negative breast cancer involves treating the human with a therapeutically effective amount of salubrinal.

16. The method of claim 14, wherein treating a human exhibiting symptoms of triple negative breast cancer involves treating the human with a therapeutically effective amount of guanabenz.

17. The method of claim 14, wherein treating a human exhibiting symptoms of triple negative breast cancer involves treating the human with a therapeutically effective amount of a combination of salubrinal and guanabenz.

18. The method of claim 14, wherein treating a human with a therapeutically effective amount of a pharmaceutically acceptable agent selected from the group consisting of salubrinal, guanabenz and a combination thereof involves administering the agent by ingestion.

19. The method of claim 14, wherein treating a human with a therapeutically effective amount of a pharmaceutically acceptable agent selected from the group consisting of salubrinal, guanabenz and a combination thereof involves administering the agent by a method selected from the group consisting of injection, osmotic pump, IV administration, dermal application, and inhalation.

20. An in vivo method for reducing the development of osteoclastogenesis comprising:
(a) providing a human having a condition that promotes osteoclastogenesis
(b) treating the human with a therapeutically effective amount of a pharmaceutically acceptable agent selected from the group consisting of salubrinal, guanabenz and a combination thereof, wherein upon treatment the development of osteoclastogenesis is reduced.

21. An in vivo method for reducing bone metastasis comprising:
(a) providing a human having a condition that promotes bone metastasis;
(b) treating the human with a therapeutically effective amount of a pharmaceutically acceptable agent selected from the group consisting of salubrinal, guanabenz and a combination thereof, wherein upon treatment bone metastasis is reduced.
